(19) United States
(12) Patent Application Publication
(10) Pub. No.: US 2008/0214642 A1
(43) Pub. Date: Sep. 4, 2008

(54) Fungicidal N-Benzyl-5-Hydroxy-5-Phenylpryrazolines, Processes For Their Preparation and Compositions Comprising Them

(76) Inventors: Markus Gewehr, Kastellaun (DE); Jochen Dietz, Mannheim (DE); Thomas Grote, Wachenheim (DE); Andreas Gypser, Mannheim (DE); Matthias Niedenbruck, Limburgerhof (DE)

Correspondence Address: HUTCHISON LAW GROUP PLLC, PO BOX 31686, RALEIGH, NC, 27612, US

(21) Appl. No.: 11/916,233

(22) PCT Filed: May 24, 2006
(86) PCT No.: PCT/EP2006/062585

§ 371 Date: Nov. 30, 2007

(30) Foreign Application Priority Data
May 31, 2005 (DE) 10 2005 025 378.4

(51) Int. Cl. A01N 43/56; C07D 231/08; A01P 3/00

(52) U.S. Cl. 514/407; 548/369.4

(57) ABSTRACT

N-benzoyl-5-hydroxy-5-phenylpyrazolines of the formula I

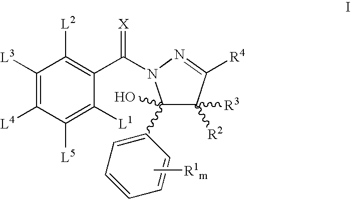

in which the substituents are as defined below:
L¹, L² are hydrogen, halogen, haloalkyl, alkenyl, haloalkenyl, alkynyl or haloalkynyl;
L³, L⁴, L⁵ are alkyl or one of the groups mentioned under L¹ and L²;
X is oxygen or sulfur;
R¹ is halogen, nitro, cyano, NR′₂, alkyl, haloalkyl, alkoxy, alkenyl, haloalkyl, alkynyl, haloalkynyl, cycloalkyl, cycloalkenyl, cycloalkynyl, phenyl, 5- or 6-membered heterocyclyl or hetaryl which contains one to four heteroatoms from the group consisting of O, N and S;
   R′ independently of one another are hydrogen or alkyl;
m is 1, 2, 3, 4 or 5;
R² is hydrogen or alkyl;
R³ is hydrogen, nitro, cyano, alkyl, haloalkyl, alkoxy, haloalkoxy, alkenyl, haloalkenyl, alkynyl, haloalkynyl or NR′₂, where
R⁴ is hydrogen, halogen, nitro, cyano, NR′₂, alkyl, haloalkyl, COOR′ or 5- or 6-membered heterocyclyl or hetaryl which contains one to four heteroatoms from the group consisting of O, N and S;
   where the variables mentioned above may be substituted according to the description;
   processes for their preparation, their use for controlling harmful fungi and compositions comprising them.

The present invention relates to N-benzyl-5-hydroxy-5-phenylpyrazolines of the formula I

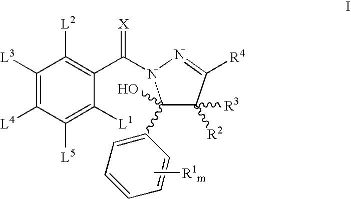

in which the substituents are as defined below:
L¹, L² independently of one another are hydrogen, halogen, C₁-C₆-haloalkyl, C₂-C₆-alkenyl, C₂-C₆-haloalkenyl, C₃-C₆-alkynyl or C₃-C₆-haloalkynyl;
L³, L⁴, L⁵ independently of one another are C₁-C₆-alkyl or one of the groups mentioned under L¹ and L²;
   where at least one of the groups mentioned above is not hydrogen;
X is oxygen or sulfur;
R¹ is halogen, nitro, cyano, NR′₂, C₁-C₆-alkyl, C₁-C₆-haloalkyl, C₁-C₆-alkoxy, C₂-C₆-alkenyl, C₂-C₆-haloalkyl, C₃-C₆-alkynyl, C₃-C₆-haloalkynyl; C₃-C₆-cycloalkyl, C₃-C₆-cycloalkenyl, C₃-C₆-cycloalkynyl, phenyl, 5- or 6-membered heterocyclyl or hetaryl which contains one to four heteroatoms from the group consisting of O, N and S;
   R′ independently of one another are hydrogen or C₁-C₄-alkyl;
m is 1, 2, 3, 4 or 5;
R² is hydrogen or C₁-C₁₀-alkyl;
R³ is hydrogen, nitro, cyano, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl or NR′₂;
R⁴ is hydrogen, halogen, nitro, cyano, NR′₂, C₁-C₄-alkyl, C₁-C₄-haloalkyl, COOR′, phenyl or 5- or 6-membered hetaryl or heterocyclyl which contains one to four heteroatoms from the group consisting of O, N and S;
   where the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₆-alkenyl, C₂-C₈-haloalkenyl, C₃-C₆-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle which contains one to four heteroatoms from the group consisting of O, N and S, CRⁱⁱⁱ═NOR^{iv}, where
   Rⁱⁱⁱ is hydrogen, alkyl, cycloalkyl or aryl and
   R^{iv} is alkyl, alkenyl, haloalkenyl, alkynyl or arylalkyl, or
   NR^{v}—CO-D-R^{vi}, where
   R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
   R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl and D is a direct bond, oxygen or nitrogen, where the nitrogen may carry one of the groups mentioned under R^{vi},
   where the aliphatic, alicyclic or aromatic groups R^{a} for their part may be partially or fully halogenated or may carry one to three groups R^{b}:
   R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups mentioned in these radicals contain 2 to 8 carbon atoms;
      and/or one to three of the following radicals:
      cycloalkyl, cycloalkoxy, heterocyclyl, heterocyclyloxy, where the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, where the hetaryl groups contain 5 or 6 ring members, and the cyclic systems may be partially or fully halogenated or substituted by alkyl or haloalkyl groups.

Moreover, the invention relates to processes for their preparation, to their use for controlling harmful fungi and to compositions comprising them.

Substituted pyrazolin-5-ones having herbicidal and fungicidal activity are known from DE-A 37 28 278, fungicidally active 3-arylpyrazoles are disclosed in WO-A 94/29276 and WO 00/20399.

However, in many cases their activity is unsatisfactory. Accordingly, it was an object of the present invention to provide compounds having improved activity.

We have found that this object is achieved by the novel 5-hydroxypyrazoles of the formula I. The novel compounds differ from those known from WO 00/20399 essentially by the substitution of the phenyl groups.

Compounds of the formula I are present in a tautomeric equilibrium with the open-chain form Ia [cf.: J. Org. Chem. USSR (1983), 2037; ibid. (1984), 1247].

The invention therefore relates to both forms, even if, for the sake of clarity, only the ring form I is mentioned in each case.

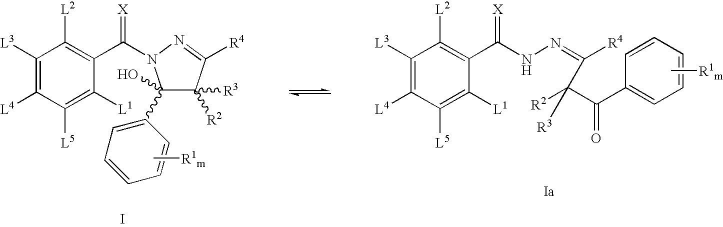

Compounds of the formula I in which X is oxygen (formula I.A) can be obtained, for example, by the following route:

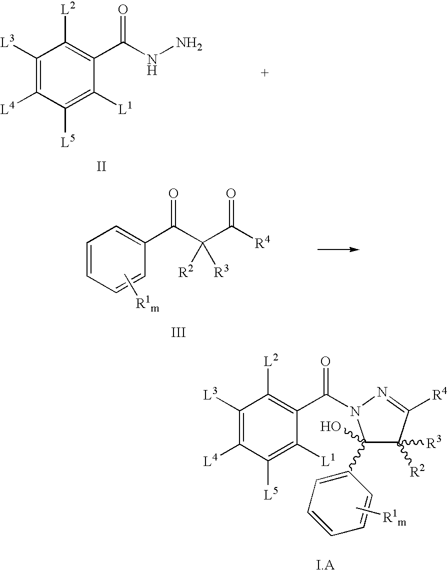

This reaction is usually carried out at from 0° C. to 200° C., preferably from 20° C. to 100° C., in an inert organic solvent [J. Org. Chem. USSR (Engl. Transl.), 16 (1980), 371; ibid. 21 (1985), 2279; ibid. 22 (1986), 250; ibid. 23 (1987), 1291; Indian J. Chem. Sect. B, 29 (1990), 887; Bull. Soc. Chem. Jp. 62 (1989), 3409].

Suitable solvents are aliphatic hydrocarbons, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane and tetrahydrofuran, nitriles, such as acetonitrile and propionitrile, alcohols, such as methanol, ethanol, n-propanol, isopropanol, n-butanol and tert-butanol, and also dimethyl sulfoxide, dimethylformamide and dimethylacetamide, particularly preferably methanol, ethanol and tetrahydrofuran. It is also possible to use mixtures of the abovementioned solvents.

The starting materials are generally reacted with one another in equimolar amounts. In terms of yield, it may be advantageous to employ an excess of III, based on II.

The hydrazides of the formula II required for preparing the compounds I are known from the literature [cf. J. Heterocycl. Chem. 16 (1976), 561; Helv. Chim. Acta, 27 (1944), 883; J. Chem. Soc. (1943), 413], or they can be prepared in accordance with the literature cited.

Hydrazides of the formula II are usually prepared from the corresponding benzoic esters of the formula V by reaction with hydrazine hydrate. In the formula V, R′ is C₁-C₄-alkyl.

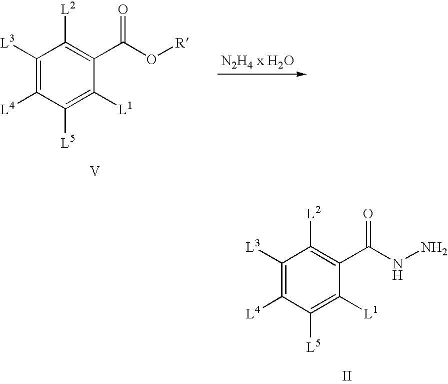

This reaction is usually carried out at from 0° C. to 150° C., preferably from 20° C. to 100° C., in an inert organic solvent [cf. J. Heterocycl. Chem. 16 (1976), 561; Helv. Chim. Acta, 27 (1944), 883; J. Chem. Soc. (1943), 413].

The diketones of the formula III required for preparing the compounds I are also known from the literature [Organikum, VEB Verlag der Wissenschaften, 15th ed. p. 584ff., Berlin 1976], or they can be prepared in accordance with the literature cited.

The compounds of the formula I in which X is sulfur (formula I.B) can be obtained from the corresponding compounds of the formula I.A by reaction with a sulfurizing agent.

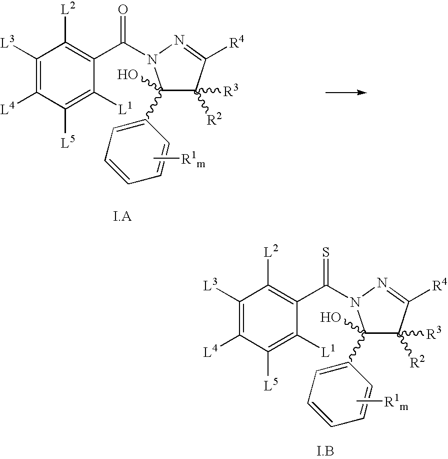

The sulfurization of I.A is carried out under conditions known per se, usually at from 0° C. to 180° C., preferably from 20° C. to 140° C., in an inert organic solvent [cf. Liebigs Ann. Chem., (1989), 177].

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, particularly preferably toluene and tetrahydrofuran. It is also possible to employ mixtures of the abovementioned solvents.

Suitable sulfurizing agents are, for example, phosphorus pentasulfide or Lawesson's reagent.

The reaction mixtures are worked up in a customary manner, for example by mixing with water, phase separation and, if appropriate, chromatographic purification of the crude products. Some of the intermediates and end products are obtained in the form of colorless or slightly brownish, viscous oils, which are purified or freed from volatile components under reduced pressure and at moderately elevated temperature. If the intermediates and end products are obtained as solids, purification can also be carried out by recrystallization or digestion.

In the definitions of the symbols given in the above formulae, collective terms were used which generally represent the following substituents:

halogen: fluorine, chlorine, bromine and iodine;
alkyl: saturated, straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 10 carbon atoms (as mentioned above), where the hydrogen atoms in these groups may be partially or fully replaced by halogen atoms as mentioned above, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;
alkenyl: unsaturated, straight-chain or branched hydrocarbon radicals having 2 to 4, 6, 8 or 10 carbon atoms and a double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated, straight-chain or branched hydrocarbon radicals having 2 to 10 carbon atoms and a double bond in any position (as mentioned above), where the hydrogen atoms in these groups may be partially or fully replaced by halogen atoms as mentioned above, in particular by fluorine, chlorine and bromine;
alkynyl: straight-chain or branched hydrocarbon groups having 2 to 4, 6, 8 or 10 carbon atoms and a triple bond in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
haloalkynyl: unsaturated, straight-chain or branched hydrocarbon radicals having 2 to 10 carbon atoms and a triple bond in any position (as mentioned above), where the hydrogen atoms in these groups may be partially or fully replaced by halogen atoms as mentioned above, in particular fluorine, chlorine and bromine;
cycloalkyl: monocyclic, saturated hydrocarbon groups having 3 to 6, 8, 10 or 12 carbon ring members, for example C₃-C₈-cycloalkyl such as cyclopropyl, cyclobutyl, cyclopentyl, cyclohexyl, cycloheptyl and cyclooctyl;
heterocyclyl: 5- or 6-membered heterocycles containing, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
hetaryl:
5-membered hetaryl containing one to four nitrogen atoms or one to three nitrogen atoms and one sulfur or oxygen atom: hetaryl groups having 5 ring members which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered hetaryl containing one to three nitrogen atoms or one nitrogen atom and one oxygen or sulfur atom: hetaryl groups having 5 ring members which, in addition to carbon atoms, may contain one to four nitrogen atoms or one to three nitrogen atoms and one sulfur or oxygen atom as ring members and in which two adjacent carbon ring members or one nitrogen and one adjacent carbon ring member may be bridged by a buta-1,3-diene-1,4-diyl group;
5-membered hetaryl which is linked via nitrogen and contains one to four nitrogen atoms, or benzo-fused 5-membered hetaryl which is linked via nitrogen and contains one to three nitrogen atoms: hetaryl groups having 5 ring members which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group, these rings being linked to the skeleton via one of the nitrogen ring members;
6-membered hetaryl containing one to three or one to four nitrogen atoms: hetaryl groups having 6 ring members which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In general, the alkyl groups in the various substituents preferably have 1 to 6 carbon atoms, in particular 1 to 4 carbon atoms, and the alkenyl or alkynyl groups mentioned contain 2 to 8 carbon atoms.

With respect to the intended use of the 5-hydroxypyrazolines of the formula I, particular preference is given to the following meanings of the substituents, in each case both on their own or in combination:

Compounds I.A are a preferred embodiment.

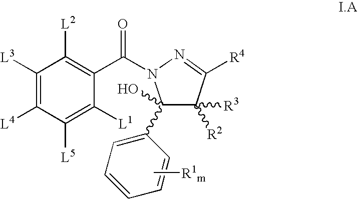

In addition, particular preference is given to compounds I in which L¹ and L² are hydrogen or halogen.

A further preferred embodiment of the compounds I relates to those where L¹, L² and L⁴ are hydrogen and L³ is not hydrogen.

A further preferred embodiment of the compounds I relates to those where L¹, L² and L³ are hydrogen and L⁴ is not hydrogen.

Particular preference is given to compounds I in which R¹ₘ is a substituent in the para position.

Special preference is also given to compounds I in which R¹ is halogen or C₁-C₄-alkyl.

Compounds of the formula I in which R² is hydrogen or C₁-C₄-alkyl, in particular hydrogen, are a preferred subject matter of the invention.

Furthermore, particular preference is given to compounds I in which R³ is hydrogen.

Moreover, particular preference is given to compounds I in which R⁴ is hydrogen or methyl.

Particular preference is likewise given to compounds I in which R⁴ is trifluoromethyl.

Special preference is also given to compounds I in which R⁴ is cyano, C₂-C₄-alkyl or C₂-C₄-haloalkyl.

With respect to their use, particular preference is given to the compounds I compiled in the tables below. Moreover, the groups mentioned for a substituent in the tables are, by themselves and independently of the combination in which they are mentioned, a particularly preferred embodiment of the substituent in question.

### Table 1

Compounds of the formula I.A in which R¹ₘ is 4-chloro, R² and R³ are hydrogen, R⁴ is methyl and the combination of L¹, L², L³, L⁴ and L⁵ for a compound corresponds in each case to one row of Table A

### Table 2

Compounds of the formula I.A in which R¹ₘ is 4-chloro, R³ is hydrogen, R² and R⁴ are methyl and the combination of L¹, L², L³, L⁴ and L⁵ for a compound corresponds in each case to one row of Table A

### Table 3

Compounds of the formula I.A in which R¹ₘ is 4-chloro, R² and R³ are hydrogen, R⁴ is trifluoromethyl and the combination of L¹, L², L³, L⁴ and L⁵ for a compound corresponds in each case to one row of Table A

### Table 4

Compounds of the formula I.A in which R¹ₘ is 4-chloro, R² is hydrogen, R³ is methyl, R⁴ is trifluoromethyl and the combination of L¹, L², L³, L⁴ and L⁵ for a compound corresponds in each case to one row of Table A

### Table 5

Compounds of the formula I.A in which R¹ₘ is 4-fluoro, R² and R³ are hydrogen, R⁴ is methyl and the combination of L¹, L², L³, L⁴ and L⁵ for a compound corresponds in each case to one row of Table A

### Table 6

Compounds of the formula I.A in which R¹ₘ is 4-fluoro, R³ is hydrogen, R² and R⁴ are methyl and the combination of L¹, L², L³, L⁴ and L⁵ for a compound corresponds in each case to one row of Table A

### Table 7

Compounds of the formula I.A in which R¹ₘ is 4-fluoro, R² and R³ are hydrogen, R⁴ is trifluoromethyl and the combination of L¹, L², L³, L⁴ and L⁵ for a compound corresponds in each case to one row of Table A

### Table 8

Compounds of the formula I.A in which R¹ₘ is 4-fluoro, R² is hydrogen, R³ is methyl, R⁴ is trifluoromethyl and the combination of L¹, L², L³, L⁴ and L⁵ for a compound corresponds in each case to one row of Table A

### Table 9

Compounds of the formula I.A in which R¹ₘ is 4-methyl, R² and R³ are hydrogen, R⁴ is methyl and the combination of L¹, L², L³, L⁴ and L⁵ for a compound corresponds in each case to one row of Table A

### Table 10

Compounds of the formula I.A in which R¹ₘ is 4-methyl, R³ is hydrogen, R² and R⁴ are methyl and the combination of L¹, L², L³, L⁴ and L⁵ for a compound corresponds in each case to one row of Table A

### Table 11

Compounds of the formula I.A in which R¹ₘ is 4-methyl, R² and R³ are hydrogen, R⁴ is trifluoromethyl and the combination of L¹, L², L³, L⁴ and L⁵ for a compound corresponds in each case to one row of Table A

### Table 12

Compounds of the formula I.A in which R¹ₘ is 4-methyl, R² is hydrogen, R³ is methyl, R⁴ is trifluoromethyl and the combination of L¹, L², L³, L⁴ and L⁵ for a compound corresponds in each case to one row of Table A

TABLE A

| No. | L¹ | L² | L³ | L⁴ | L⁵ |
|---|---|---|---|---|---|
| A-1 | F | H | H | H | H |
| A-2 | H | H | F | H | H |
| A-3 | H | H | H | F | H |
| A-4 | Cl | H | H | H | H |
| A-5 | H | H | Cl | H | H |
| A-6 | H | H | H | Cl | H |
| A-7 | Br | H | H | H | H |
| A-8 | H | H | Br | H | H |
| A-9 | H | H | H | Br | H |
| A-10 | CH₃ | H | H | H | H |
| A-11 | H | H | CH₃ | H | H |
| A-12 | H | H | H | CH₃ | H |
| A-13 | CH₂CH₃ | H | H | H | H |
| A-14 | H | H | CH₂CH₃ | H | H |
| A-15 | H | H | H | CH₂CH₃ | H |
| A-16 | F | H | H | F | H |
| A-17 | H | H | F | F | H |
| A-18 | H | H | F | H | F |
| A-19 | Cl | H | H | Cl | H |
| A-20 | H | H | Cl | Cl | H |
| A-21 | H | H | Cl | H | Cl |
| A-22 | Br | H | H | Br | H |
| A-23 | H | H | Br | Br | H |
| A-24 | H | H | Br | H | Br |
| A-25 | F | H | H | Cl | H |
| A-26 | H | H | F | Cl | H |
| A-27 | H | H | F | H | Cl |
| A-28 | F | H | H | Br | H |
| A-29 | H | H | F | Br | H |
| A-30 | H | H | F | H | Br |
| A-31 | Cl | H | H | F | H |
| A-32 | H | H | Cl | F | H |
| A-33 | Cl | H | H | Br | H |
| A-34 | H | H | Cl | Br | H |
| A-35 | H | H | Cl | H | Br |
| A-36 | Br | H | H | F | H |
| A-37 | H | H | Br | F | H |
| A-38 | Br | H | H | Cl | H |
| A-39 | H | H | Br | Cl | H |

The compounds I are suitable as fungicides. They are distinguished by an outstanding effectiveness against a broad spectrum of phytopathogenic fungi from the classes of the Ascomycetes, Deuteromycetes, Oomycetes and Basidiomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes,
Aphanomyces species on sugar beet and vegetables,
Ascochyta species on cereals and vegetables,
Bipolaris and Drechslera species on corn, cereals, rice and lawns, such as, for example, D. maydis on corn,
Blumeria graminis (powdery mildew) on cereals,
Botrytis cinerea (gray mold) on strawberries, vegetables, flowers and grapevines,
Bremia lactucae on lettuce,
Cercospora species on corn, soybeans, rice and sugar beet,
Cochliobolus species on corn, cereals, rice, such as, for example Cochliobolus sativus on cereals, Cochliobolus miyabeanus on rice,
Colletotricum species on soybeans and cotton,
Drechslera species, Pyrenophora species on corn, cereals, rice and lawns, such as, for example, D. teres on barley or D. tritici-repentis on wheat,
Esca on grapevines, caused by Phaeoacremonium chlamydosporium, Ph. Aleophilum and Formitipora punctata (syn. Phellinus punctatus),
Exserohilum species on corn,
Erysiphe cichoracearum and Sphaerotheca fuliginea on cucumbers;
Fusarium and Verticillium species on various plants, such as, for example, F graminearum or F. culmorum on cereals or F. oxysporum on a multitude of plants, such as, for example, tomatoes,
Gaeumanomyces graminis on cereals,
Gibberella species on cereals and rice (for example Gibberella fujikuroi on rice),
Grainstaining complex on rice,
Helminthosporium species on corn and rice,
Michrodochium nivale on cereals,
Mycosphaerella species on cereals, bananas and groundnuts, such as, for example, M. graminicola on wheat or M. fijiensis on bananas,
Peronospora species on cabbage and bulbous plants, such as, for example, P. brassicae on cabbage or P. destructor on onions,
Phakopsara pachyrhizi and Phakopsara meibomiae on soybeans,
Phomopsis species on soybeans and sunflowers,
Phytophthora infestans on potatoes and tomatoes,
Phytophthora species on various plants, such as, for example, P. capsici on bell pepper,
Plasmopara viticola on grapevines,
Podosphaera leucotricha on apples,
Pseudocercosporella herpotrichoides on cereals,
Pseudoperonospora on various plants, such as, for example, P. cubensis on cucumber or P. humili on hops,
Puccinia species on various plants, such as, for example, P. triticina, P. striformins, P. hordei or P. graminis on cereals or P. asparagi on asparagus,
Pyricularia oryzae, Corticium sasakii, Sarocladium oryzae, S. attenuatum, Entyloma oryzae on rice,
Pyricularia grisea on lawns and cereals,
Pythium spp. on lawns, rice, corn, cotton, oilseed rape, sunflowers, sugar beet, vegetables and other plants, such as, for example, P. ultiumum on various plants, P. aphanidermatum on lawns,
Rhizoctonia species on cotton, rice, potatoes, lawns, corn, oilseed rape, potatoes, sugar beet, vegetables and on various plants, such as, for example, R. solani on beet and various plants,
Rhynchosporium secalis on barley, rye and triticale,
Sclerotinia species on oilseed rape and sunflowers,
Septoria tritici and Stagonospora nodorum on wheat,
Erysiphe (syn. Uncinula) necator on grapevines,
Setospaeria species on corn and lawns,
Sphacelotheca reilinia on corn,
Thievaliopsis species on soybeans and cotton,
Tilletia species on cereals,
Ustilago species on cereals, corn and sugar cane, such as, for example, U. maydis on corn,
Venturia species (scab) on apples and pears, such as, for example, V. inaequalis on apples.

They are particularly suitable for controlling harmful fungi from the class of the Peronosporomycetes (syn. Oomycetes) such as Peronospora species, Phytophthora species, Plasmopara viticola, Pseudoperonospora species and Pythium species.

The compounds I are also suitable for controlling harmful fungi in the protection of materials (for example wood, paper, paint dispersions, fibers or fabrics) and in the protection of stored products. In the protection of wood, particular attention is paid to the following harmful fungi:

Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds, materials or the soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of from 1 to 1000 g/100 kg, preferably from 5 to 100 g/100 kg, of seed are generally necessary.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds of the formula I may be present in various crystal modifications whose biological activities may differ. They are likewise provided by the present invention.

The compounds I can be converted into the customary formulations, for example solutions, emulsions, suspensions, dusts, powders, pastes and granules. The use form depends on the particular intended use; it should in each case ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for broadcasting and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved in 90 parts by weight of water or in a water-soluble solvent. As an alternative, wetting agents or other auxiliaries are added. The active compound dissolves upon dilution with water. In this way, a formulation having a content of 10% by weight of active compound is obtained.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetting agents and silica gel. Dilution with water gives a stable dispersion or solution of the active compound. The active compound content of the formulation is 75% by weight.

### H Gel Formulations (GF)

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

### I Dustable Powders (DP, DS)

5 parts by weight of the active compounds are ground finely and mixed intimately with 95 parts by weight of finely divided kaolin. This gives a dustable product having an active compound content of 5% by weight.

### J Granules (GR, FG, GG, MG)

0.5 part by weight of the active compounds is ground finely and associated with 99.5 parts by weight of carriers. Current methods are extrusion, spray-drying and the fluidized bed. This gives granules to be applied undiluted having an active compound content of 0.5% by weight.

### K ULV Solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or of the use forms prepared therefrom, e.g. in the form of directly sprayable solutions, powders, suspensions or dispersions, emulsions, oil dispersions, pastes, dustable products, materials for broadcasting or granules, by means of spraying, atomizing, dusting, broadcasting or watering. The use forms depend entirely on the intended purposes; they should always ensure the finest possible distribution of the active compounds according to the invention.

Aqueous use forms can be prepared from emulsion concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, it is also possible to prepare concentrates comprising active substance, wetting agent, tackifier, dispersant or emulsifier and possibly solvent or oil which are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even the active compound without additives.

Oils of various types, wetting agents, adjuvants, herbicides, fungicides, other pesticides and bactericides can be added to the active compounds, if appropriate also not until immediately before use (tank mix). These agents can be added to the preparations according to the invention in a weight ratio of 1:100 to 100:1, preferably of 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the use form as fungicides, also be present together with other active compounds, e.g. with herbicides, insecticides, growth regulators, fungicides or also with fertilizers. When mixing the compounds (I) or the compositions comprising them with one or more further active compounds, in particular fungicides, it is in many cases possible to widen the activity spectrum or to prevent the development of resistance. In many cases, synergistic effects are obtained.

The following list of fungicides, with which the compounds according to the invention can be used in conjunction, is intended to illustrate the possible combinations but does not limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

The procedure described in the following synthesis example was used to prepare further compounds I by appropriate modification of the starting materials. The compounds obtained in this manner are listed in the table below, together with physical data.

### Example 1

### Preparation of (4-chlorophenyl)-[5-(2,5-difluorophenyl)-5-hydroxy-3-trifluoromethyl-4,5-dihydropyrazol-1-yl]methanone [1-6]

A solution of 0.68 g of 4-chlorobenzoic acid hydrazide and 1.01 g of 1-(2,5-difluorophenyl)-4,4,4-trifluoro-1,3-butanedione in 20 ml of ethanol was stirred at 80° C. for 8 hours. The reaction mixture was freed from the solvent and purified by chromatography on silica gel. This gave 0.30 g of the title compound as colorless crystals of melting point 110-113° C.

### Example 2

### Preparation of (3-aminophenyl)-[5-(4-chloro-2,5-difluorophenyl)-5-hydroxy-3-trifluoromethyl-4,5-dihydropyrazol-1-yl]methanone [1-18]

A solution of 0.61 g of 3-aminobenzoic acid hydrazide and 1.15 g of 1-(4-chloro-2,5-difluorophenyl)-4,4,4-trifluoro-1,3-butanedione in 20 ml of ethanol was stirred at 80° C. for 8 hours. The reaction mixture was freed from the solvent and purified by chromatography on silica gel. This gave 0.33 g of the title compound as colorless crystals of melting point 123-124° C.

TABLE I

| | | | | | | | | | | | Phys. Data (m.p. [° C.]; |
|---|---|---|---|---|---|---|---|---|---|---|---|
| No. | L¹ | L² | L³ | L⁴ | L⁵ | X | R¹ₘ | R² | R³ | R⁴ | ¹H-NMR (CDCl₃ δ [ppm]) |
| I-1 | H | H | H | CH₃ | H | O | 2,4-F₂ | H | H | CF₃ | 163-166 |
| I-2 | H | H | H | Br | H | O | 2,4-F₂ | H | H | CF₃ | 134-136 |
| I-3 | H | H | H | Cl | H | O | 2,4-F₂ | H | H | CF₃ | 7.7 (m); 7.4 (m); 6.9 (m); 6.75 (m); |
| | | | | | | | | | | | 5.5 (s); 3.4 (m) |
| I-4 | H | H | H | CH₃ | H | O | 2,5-F₂ | H | H | CF₃ | 7.75 (d); 7.45 (m); 7.2 (d); 7.0 (m); |
| | | | | | | | | | | | 5.6 (s); 3.2 (m); 2.2 (s) |
| I-5 | H | H | H | Br | H | O | 2,5-F₂ | H | H | CF₃ | 7.7 (d); 7.55 (d); 7.45 (m); 7.05 (m); |
| | | | | | | | | | | | 5.5 (s); 3.2 (m) |
| I-6 | H | H | H | Cl | H | O | 2,5-F₂ | H | H | CF₃ | 110-113 |
| I-7 | H | H | CH₃ | H | H | O | 2,5-F₂ | H | H | CF₃ | 115-118 |
| I-8 | H | H | Cl | H | H | O | 2,5-F₂ | H | H | CF₃ | 7.8 (s); 7.75 (d); 7.45 (m); 7.3 (m); |
| | | | | | | | | | | | 7.0 (m); 5.5 (s); 3.4 (m) |
| I-9 | H | H | CH₃ | H | H | O | 2,4-F₂ | H | H | CF₃ | 163-165 |
| I-10 | H | H | Cl | H | H | O | 2,4-F₂ | H | H | CF₃ | 130-133 |
| 1-11 | H | Cl | H | H | H | O | 2,4-F₂ | H | H | CF₃ | 7.7 (m); 7.4 (m); 6.95 (m); 5.25 (s); |
| | | | | | | | | | | | 3.2 (m) |
| 1-12 | H | Cl | H | Cl | H | O | 2,4-F₂ | H | H | CF₃ | 7.7 (m); 7.45 (s); 7.2 (m); 6.9 (m); |
| | | | | | | | | | | | 5.4 (s); 3.45 (m) |
| 1-13 | H | Cl | H | Cl | H | O | 2,5-F₂ | H | H | CF₃ | 7.5 (m); 7.25 (m); 7.1 (m); 5.2 (s); |
| | | | | | | | | | | | 3.45 (m) |
| 1-14 | H | CF₃ | H | F | H | O | 2,5-F₂ | H | H | CF₃ | 7.45 (m); 7.25 (m); 7.05 (m); |
| | | | | | | | | | | | 5.05 (s); 3.3 (m) |
| 1-15 | H | H | H | Cl | H | O | 5-Cl, 2,5-F₂ | H | H | CF₃ | 164-166 |
| 1-16 | H | H | H | Cl | H | O | 2-F, 4-Cl, 5-OCH₃ | H | H | CF₃ | 210-213 |
| 1-17 | H | H | Cl | H | H | O | 5-Cl, 2,5-F₂ | H | H | CF₃ | 145-147 |
| 1-18 | H | H | NH₂ | H | H | O | 5-Cl, 2,5-F₂ | H | H | CF₃ | 123-124 |
| 1-19 | H | H | CH₃ | H | H | O | 3-CH₃, 4-Cl | H | H | CF₃ | 7.8 (m); 7.45 (s); 7.35 (m); 6.7 (s); |
| | | | | | | | | | | | 3.6 (m); 2.45 (s); 2.35 (s) |
| 1-20 | H | H | Cl | H | H | O | 3-CH₃, 4-Cl | H | H | CF₃ | 8.0 (s); 7.9 (d); 7.4 (m); 6.55 (s); |
| | | | | | | | | | | | 3.6 (m); 2.4 (s) |
| 1-21 | H | Cl | H | Cl | H | O | 3-CH₃, 4-Cl | H | H | CF₃ | 122-123 |
| 1-22 | H | Cl | H | H | H | O | 5-Cl, 2,5-F₂ | H | H | CF₃ | 7.7 (m); 7.6 (m); 7.4 (m); 7.2 (m); |
| | | | | | | | | | | | 5.5 (s); 3.45 (m) |

### Examples of the Action Against Harmful Fungi

The fungicidal action of the compounds of the formula I was demonstrated by the following tests:

The active compounds were prepared as a stock solution comprising 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent/emulsifier of 99 to 1. The mixture was then made up with water to 100 ml. This stock solution was diluted with the solvent/emulsifier/water mixture described to the concentration of active compounds stated below.

### Use Example 1

### Curative Activity Against Brown Rust of Wheat Caused by Puccinia recondita

Leaves of potted wheat seedlings of the cultivar “Kanzler” were inoculated with a spore suspension of brown rust (Puccinia recondita). The pots were then placed in a chamber with high atmospheric humidity (90 to 95%) and at 20 to 22° C. for 24 hours. During this time, the spores germinated and the germ tubes penetrated into the leaf tissue. The next day, the infected plants were sprayed to run-off point with the active compound solution described above at the active compound concentration stated below. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

In this test, the plants which had been treated with 250 ppm of the active compound I-2, I-9 or I-14 showed an infection of not more than 5%, whereas the untreated plants were 90% infected.

### Use Example 2

### Curative Activity Against Soybean Rust Caused by Phakopsora pachyrhizi

Leaves of potted soybean seedlings of the cultivar “Oxford” were inoculated with a spore suspension of soybean rust (Phakpsora pachyrhizi). The pots were then placed into a chamber with high atmospheric humidity (90 to 95%) and at 23 to 27° C. for 24 hours. During this time, the spores germinated and the germ tubes penetrated into the leaf tissue. After 1 day, the infected plants were sprayed to run-off point with the active compound solution described above at the active compound concentration stated below. After the spray coating had dried on, the test plants were cultivated in a greenhouse at temperatures between 23 and 27° C. and at 60 to 80% relative atmospheric humidity for 14 days. The extent of the rust fungus development on the leaves was then determined.

In this test, the plants which had been treated with 1000 ppm of the active compound I-17 showed an infection of 3%, whereas the untreated plants were 90% infected.

1-9. (canceled)

10. A compound of formula I, 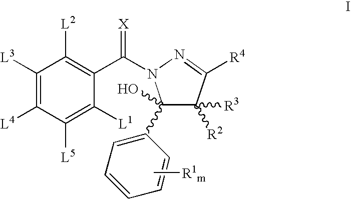
wherein,
L¹, L² independently of one another are hydrogen, halogen, C₁-C₆-haloalkyl, C₂-C₆-alkenyl, C₂-C₆-haloalkenyl, C₃-C₆-alkynyl or C₃-C₆-haloalkynyl;
L³, L⁴, L⁵ independently of one another are C₁-C₆-alkyl or one of the groups mentioned under L¹ and L²;
provided that at least one of the groups mentioned above is not hydrogen;
X is oxygen or sulfur;
R¹ is halogen, nitro, cyano, NR′₂, C₁-C₆-alkyl, C₁-C₆-haloalkyl, C₁-C₆-alkoxy, C₂-C₆-alkenyl, C₂-C₆-haloalkyl, C₃-C₆-alkynyl, C₃-C₆-haloalkynyl; C₃-C₆-cycloalkyl, C₃-C₆-cycloalkenyl, C₃-C₆-cycloalkynyl, phenyl, 5- or 6-membered heterocyclyl or hetaryl which contains one to four heteroatoms from the group consisting of O, N and S;
R′ independently of one another are hydrogen or C₁-C₄-alkyl;
m is 1, 2, 3, 4 or 5;
R² is hydrogen or C₁-C₁₀-alkyl;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl or C₂-C₄-haloalkynyl, wherein
R⁴ is hydrogen, halogen, nitro, cyano, NR′₂, C₁-C₄-alkyl, C₁-C₄-haloalkyl, COOR′, phenyl or 5- or 6-membered heterocyclyl or hetaryl which contains one to four heteroatoms from the group consisting of O, N and S;
wherein the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle which contains one to four heteroatoms from the group consisting of O, N and S,
CRⁱⁱⁱ═NOR^{iv}, wherein
Rⁱⁱⁱ is hydrogen, alkyl, cycloalkyl or aryl and
R^{iv} is alkyl, alkenyl, haloalkenyl, alkynyl or arylalkyl, or NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl and D is a direct bond, oxygen or nitrogen, wherein the nitrogen may carry one of the groups mentioned under R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms and the alkenyl or alkynyl groups mentioned in these radicals contain 2 to 8 carbon atoms; one to three of the following radicals, cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and
the cyclic systems may be partially or fully halogenated or substituted by alkyl or haloalkyl groups.

11. The compound of claim 10, wherein said compound has the following structure I.A,

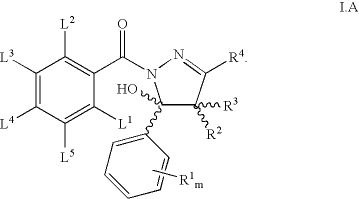

12. The compound of claim 11, wherein each of L¹, L² and L³ is hydrogen and L⁴ is other than hydrogen.

13. The compound of claim 10, wherein each of L¹, L² and L³ is hydrogen and L⁴ is other than hydrogen.

14. A process for preparing a compound of formula I.A 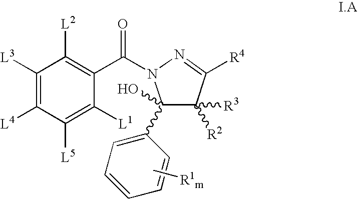
wherein,
L¹, L² independently of one another are hydrogen, halogen, C₁-C₆-haloalkyl, C₂-C₆-alkenyl, C₂-C₆-haloalkenyl, C₃-C₆-alkynyl or C₃-C₆-haloalkynyl;
L³, L⁴, L⁵ independently of one another are C₁-C₆-alkyl or one of the groups mentioned under L¹ and L²;
provided that at least one of the groups mentioned above is not hydrogen;
R¹ is halogen, nitro, cyano, NR′₂, C₁-C₆-alkyl, C₁-C₆-haloalkyl, C₁-C₆-alkoxy, C₂-C₆-alkenyl, C₂-C₆-haloalkyl, C₃-C₆-alkynyl, C₃-C₆-haloalkynyl; C₃-C₆-cycloalkyl, C₃-C₆-cycloalkenyl, C₃-C₆-cycloalkynyl, phenyl, 5- or 6-membered heterocyclyl or hetaryl which contains one to four heteroatoms from the group consisting of O, N and S;
R′ independently of one another are hydrogen or C₁-C₄-alkyl;
m is 1, 2, 3, 4 or 5;
R² is hydrogen or C₁-C₁₀-alkyl;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl or C₂-C₄-haloalkynyl, wherein
R⁴ is hydrogen, halogen, nitro, cyano, NR′₂, C₁-C₄-alkyl, C₁-C₄-haloalkyl, COOR′, phenyl or 5- or 6-membered heterocyclyl or hetaryl which contains one to four heteroatoms from the group consisting of O, N and S;
wherein the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle which contains one to four heteroatoms from the group consisting of O, N and S,
CRⁱⁱⁱ═NOR^{iv}, wherein
Rⁱⁱⁱ is hydrogen, alkyl, cycloalkyl or aryl and
R^{iv} is alkyl, alkenyl, haloalkenyl, alkynyl or arylalkyl, or NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl and D is a direct bond, oxygen or nitrogen, wherein the nitrogen may carry one of the groups mentioned under R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms and the alkenyl or alkynyl groups mentioned in these radicals contain 2 to 8 carbon atoms; one to three of the following radicals, cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and
the cyclic systems may be partially or fully halogenated or substituted by alkyl or haloalkyl groups;
said method comprising:
contacting a compound of formula II, 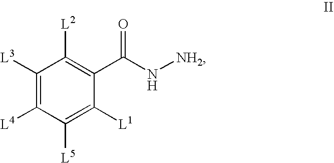
wherein L¹, L², L³, L⁴ and R⁵ are as defined above with a compound of formula III, 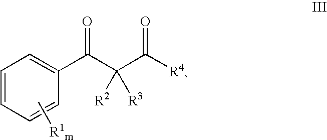
wherein m, R¹, R², R³ and R⁴ are as defined above,
wherein a compound of formula I.A is prepared.

15. A fungicidal composition comprising a solid or liquid carrier and a compound of the formula I, 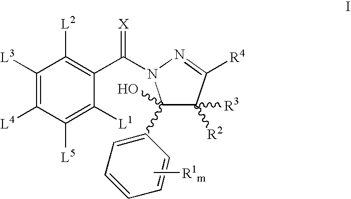
wherein,
L¹, L² independently of one another are hydrogen, halogen, C₁-C₆-haloalkyl, C₂-C₆-alkenyl, C₂-C₆-haloalkenyl, C₃-C₆-alkynyl or C₃-C₆-haloalkynyl;
L³, L⁴, L⁵ independently of one another are C₁-C₆-alkyl or one of the groups mentioned under L¹ and L²;
provided that at least one of the groups mentioned above is not hydrogen;
X is oxygen or sulfur;
R¹ is halogen, nitro, cyano, NR′₂, C₁-C₆-alkyl, C₁-C₆-haloalkyl, C₁-C₆-alkoxy, C₂-C₆-alkenyl, C₂-C₆-haloalkyl, C₃-C₆-alkynyl, C₃-C₆-haloalkynyl; C₃-C₆-cycloalkyl, C₃-C₆-cycloalkenyl, C₃-C₆-cycloalkynyl, phenyl, 5- or 6-membered heterocyclyl or hetaryl which contains one to four heteroatoms from the group consisting of O, N and S;
R′ independently of one another are hydrogen or C₁-C₄-alkyl;
m is 1, 2, 3, 4 or 5;
R² is hydrogen or C₁-C₁₀-alkyl;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl or C₂-C₄-haloalkynyl, wherein
R⁴ is hydrogen, halogen, nitro, cyano, NR′₂, C₁-C₄-alkyl, C₁-C₄-haloalkyl, COOR′, phenyl or 5- or 6-membered heterocyclyl or hetaryl which contains one to four heteroatoms from the group consisting of O, N and S;
wherein the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle which contains one to four heteroatoms from the group consisting of O, N and S,
CRⁱⁱⁱ═NOR^{iv}, wherein
Rⁱⁱⁱ is hydrogen, alkyl, cycloalkyl or aryl and
R^{iv} is alkyl, alkenyl, haloalkenyl, alkynyl or arylalkyl, or NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl and D is a direct bond, oxygen or nitrogen, wherein the nitrogen may carry one of the groups mentioned under R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms and the alkenyl or alkynyl groups mentioned in these radicals contain 2 to 8 carbon atoms; one to three of the following radicals, cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and
the cyclic systems may be partially or fully halogenated or substituted by alkyl or haloalkyl groups.

16. The composition of claim 15, wherein said compound has the following structure I.A,

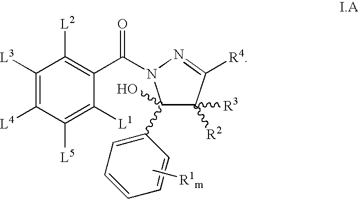

17. The composition of claim 16, wherein each of L¹, L² and L³ is hydrogen and L⁴ is other than hydrogen.

18. The composition of claim 15, wherein each of L¹, L² and L³ is hydrogen and L⁴ is other than hydrogen.

19. The composition of claim 15, further comprising another active compound.

20. A seed comprising, a compound of the formula I, 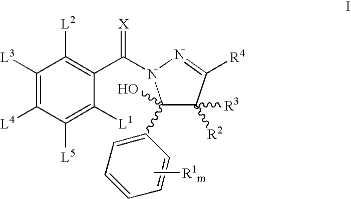
wherein,
L¹, L² independently of one another are hydrogen, halogen, C₁-C₆-haloalkyl, C₂-C₆-alkenyl, C₂-C₆-haloalkenyl, C₃-C₆-alkynyl or C₃-C₆-haloalkynyl;
L³, L⁴, L⁵ independently of one another are C₁-C₆-alkyl or one of the groups mentioned under L¹ and L²;
provided that at least one of the groups mentioned above is not hydrogen;
X is oxygen or sulfur;
R¹ is halogen, nitro, cyano, NR′₂, C₁-C₆-alkyl, C₁-C₆-haloalkyl, C₁-C₆-alkoxy, C₂-C₆-alkenyl, C₂-C₆-haloalkyl, C₃-C₆-alkynyl, C₃-C₆-haloalkynyl; C₃-C₆-cycloalkyl, C₃-C₆-cycloalkenyl, C₃-C₆-cycloalkynyl, phenyl, 5- or 6-membered heterocyclyl or hetaryl which contains one to four heteroatoms from the group consisting of O, N and S;
R′ independently of one another are hydrogen or C₁-C₄-alkyl;
m is 1, 2, 3, 4 or 5;
R² is hydrogen or C₁-C₁₀-alkyl;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl or C₂-C₄-haloalkynyl, wherein
R⁴ is hydrogen, halogen, nitro, cyano, NR′₂, C₁-C₄-alkyl, C₁-C₄-haloalkyl, COOR′, phenyl or 5- or 6-membered heterocyclyl or hetaryl which contains one to four heteroatoms from the group consisting of O, N and S;
wherein the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle which contains one to four heteroatoms from the group consisting of O, N and S,
CRⁱⁱⁱ═NOR^{iv}, wherein
Rⁱⁱⁱ is hydrogen, alkyl, cycloalkyl or aryl and
R^{iv} is alkyl, alkenyl, haloalkenyl, alkynyl or arylalkyl, or NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl and D is a direct bond, oxygen or nitrogen, wherein the nitrogen may carry one of the groups mentioned under R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms and the alkenyl or alkynyl groups mentioned in these radicals contain 2 to 8 carbon atoms; one to three of the following radicals, cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and
the cyclic systems may be partially or fully halogenated or substituted by alkyl or haloalkyl groups
said compound being present in an amount of from 1 to 1000 g per 100 kg seed.

21. The seed of claim 20, wherein said compound has the following structure I.A,

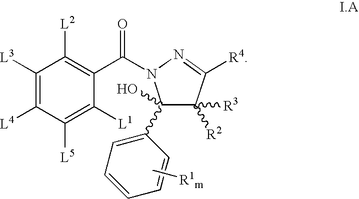

22. The seed of claim 21, wherein each of L¹, L² and L³ is hydrogen and L⁴ is other than hydrogen.

23. The seed of claim 20, wherein each of L¹, L² and L³ is hydrogen and L⁴ is other than hydrogen.

24. A method for controlling phytopathogenic harmful fungi comprising, treating the fungi or the materials, plants, the soil or seed to be protected from fungal attack with an effective amount of a compound of formula I, 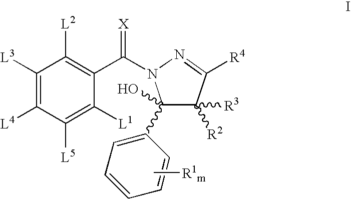
wherein,
L¹, L² independently of one another are hydrogen, halogen, C₁-C₆-haloalkyl, C₂-C₆-alkenyl, C₂-C₆-haloalkenyl, C₃-C₆-alkynyl or C₃-C₆-haloalkynyl;
L³, L⁴, L⁵ independently of one another are C₁-C₆-alkyl or one of the groups L¹ and L²;
provided that at least one of the groups mentioned above is not hydrogen;
X is oxygen or sulfur;
R¹ is halogen, nitro, cyano, NR′₂, C₁-C₆-alkyl, C₁-C₆-haloalkyl, C₁-C₆-alkoxy, C₂-C₆-alkenyl, C₂-C₆-haloalkyl, C₃-C₆-alkynyl, C₃-C₆-haloalkynyl; C₃-C₆-cycloalkyl, C₃-C₆-cycloalkenyl, C₃-C₆-cycloalkynyl, phenyl, 5- or 6-membered heterocyclyl or hetaryl which contains one to four heteroatoms from the group consisting of O, N and S;
R′ independently of one another are hydrogen or C₁-C₄-alkyl;
m is 1, 2, 3, 4 or 5;
R² is hydrogen or C₁-C₁₀-alkyl;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl or C₂-C₄-haloalkynyl, wherein
R⁴ is hydrogen, halogen, nitro, cyano, NR′₂, C₁-C₄-alkyl, C₁-C₄-haloalkyl, COOR′, phenyl or 5- or 6-membered heterocyclyl or hetaryl which contains one to four heteroatoms from the group consisting of O, N and S;
wherein the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle which contains one to four heteroatoms from the group consisting of O, N and S,
CRⁱⁱⁱ═NOR^{iv}, wherein
Rⁱⁱⁱ is hydrogen, alkyl, cycloalkyl or aryl and
R^{iv} is alkyl, alkenyl, haloalkenyl, alkynyl or arylalkyl, or NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl and D is a direct bond, oxygen or nitrogen, wherein the nitrogen may carry one of the groups mentioned under R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms and the alkenyl or alkynyl groups mentioned in these radicals contain 2 to 8 carbon atoms; one to three of the following radicals, cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and
the cyclic systems may be partially or fully halogenated or substituted by alkyl or haloalkyl groups,
wherein said phytopathogenic harmful fungi is controlled.

25. The method of claim 24, wherein said compound has the following structure I.A,

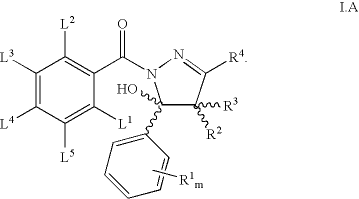

26. The method of claim 25, wherein each of L¹, L² and L³ is hydrogen and L⁴ is other than hydrogen.

27. The method of claim 24, wherein each of L¹, L² and L³ is hydrogen and L⁴ is other than hydrogen.
